(19) United States
(12) Patent Application Publication
(10) Pub. No.: US 2023/0102414 A1
(43) Pub. Date: Mar. 30, 2023

(54) METHODS, DEVICES, AND COMPOSITIONS FOR LESION REPAIR AND PREVENTION

(71) Applicant: UNIVERSITY OF FLORIDA RESEARCH FOUNDATION, INCORPORATED, Gainesville, FL (US)

(72) Inventors: Brian Lim HOH, Gainesville, FL (US); Christopher D. BATICH, Gainesville, FL (US); Koji HOSAKA, Newberry, FL (US)

(21) Appl. No.: 17/911,026

(22) PCT Filed: Mar. 11, 2021
(86) PCT No.: PCT/US2021/021945

§ 371 Date: Sep. 12, 2022

Related U.S. Application Data
(60) Provisional application No. 62/988,155, filed on Mar. 11, 2020.

(51) Int. Cl. A61K 38/19; A61K 47/34; A61K 45/06; A61K 9/00

(52) U.S. Cl. CPC A61K 38/195; A61K 38/19; A61K 47/34; A61K 45/06; A61K 9/0024

(57) ABSTRACT

The present disclosure provides for methods, devices, and compositions for controlling factors involved in the healing of lesions. The methods, devices, and composition include a first therapeutic agent which includes an amount of MCP-1 and/or osteopontin effective to promote recruitment of macrophages to the lesion site. In additional embodiments, there is further provided a second therapeutic agent which includes an inhibitor of at least one of IL-17A, CXCL1, or IL-6 to promote polarization of an amount of the macrophages to M2 phenotype macrophages at the lesion site.

### GOVERNMENT SUPPORT

This invention was made with government support under Grant Nos. NS083673, NS110710, and NS067058 awarded by the National Institutes of Health. The government has certain rights in the invention.

### FIELD

The present disclosure is directed to methods of treatment, devices, and systems for the repair of lesions in a subject via controlling components involved in the subject's internal inflammatory response.

In certain aspects, the present disclosure is directed to methods of treatment for the repair of lesions which administer a first therapeutic agent to promote recruitment of macrophages to a lesion site and a second therapeutic agent to promote conversion of the macrophages to polarized anti-inflammatory (M2 phenotype) macrophages at the lesion site.

In other aspects, the present disclosure is directed to devices and compositions for carrying out the methods of treatment.

### BACKGROUND

Generally, wound healing is characterized by an inflammatory model including injury, repair, and remodeling phases in succession. In the injury phase, circulating neutrophils are recruited by chemokines and rapidly infiltrate the area of injury. Thereafter, proinflammatory monocytes circulate and are recruited to the site of injury to remove extracellular injury. The repair and remodeling stages are characterized by repair and remodeling via the proliferation of fibroblasts, collagen, and the like. It has been found that macrophages include multiple phenotypes. For example, it has been found that the anti-inflammatory M2 phenotype macrophage promotes repair and remodeling during lesion repair. See J. Neurointerv Surg., 2018 March; 10(3): 301-305. In the context of aneurysm healing, for example, elevated M2 phenotype macrophage levels have been found in the repair and remodeling stage. Mechanisms for control of and transformation of macrophages (other than the M2 phenotype) to M2 macrophages (referred to as macrophage polarization) are still unclear. Nevertheless, if further treatments for promoting increased M2 phenotype macrophages to a lesion site were developed, it is believed such treatments could significantly enhance lesion healing, including vascular lesions, wounds, burns, and ulcers. Thus, there is currently a need for understanding and promoting the presence of M2 phenotype macrophages at a lesion site at the desired point in time to further accelerate lesion repair.

### SUMMARY

The present disclosure is thus directed to methods of treatment, devices, and systems for the repair of lesions, including vascular lesions (e.g., aneurysms), wounds, burns, or ulcers, via controlling components of a subject's inflammatory response. The solutions provided herein address the deficiency in the art regarding the identification and timing of factors critical in the prevention and repair of lesions. In certain embodiments, the general recruitment of macrophages to a lesion site in a subject is promoted by administration of a first therapeutic agent comprising monocyte chemotactic protein (MCP-1) and osteopontin to the subject. Thereafter, the conversion of the macrophages to polarized anti-inflammatory M2 phenotype macrophages useful for lesion repair are promoted at the lesion site by administration of a second therapeutic agent to the subject comprising an inhibitor of at least one of IL-17A, CXCL1, or IL-6. In this way, an anti-inflammatory environment is further promoted at the lesion site, particularly for the repair and remodeling stages of lesion repair at the appropriate point in time.

In certain embodiments and without limitation, aspects of the present disclosure are directed to the treatment, including prevention and repair, of vascular injury, such as aneurysms. To accomplish the same, in certain embodiments, the first and second therapeutic agents may be administered systemically while an aneurysm coil for the treatment of aneurysms is introduced to the aneurysm site. In this way, aneurysm healing is promoted by the therapeutic agents along with the buildup of new tissue on the coil. In other embodiments, the therapeutic agents may be included within or as a coating on the coil itself such that the therapeutic agents are released in an ordered regimen to promote lesion healing. It is understood that the principles described herein related to the treatment of aneurysms may be applied to other types of lesions, and are thus not so limited.

In accordance with an aspect of the present disclosure, there is disclosed a process of treating a lesion at a lesion site in a subject comprising:

administering an amount of a first therapeutic agent comprising MCP-1 and/or osteopontin to the subject effective to promote recruitment of macrophages to the lesion site; and

administering an amount of a second therapeutic agent comprising an inhibitor of at least one of IL-17A, CXCL1, or IL-6 effective to promote polarization of an amount of the macrophages to M2 phenotype macrophages at the lesion site.

In accordance with another aspect, there is disclosed a process for preventing formation of, enlargement of, and/or rupture of an aneurysm in a subject comprising:

locating a device comprising a polymer at a lesion site within a vasculature of the subject;

administering to the subject an amount of a first therapeutic agent comprising MCP-1 and/or osteopontin effective to promote recruitment of macrophages to the lesion site and a second therapeutic agent comprising an inhibitor of at least one of IL-17A, CXCL1, or IL-6 effective to promote polarization of an amount of the macrophages to M2 phenotype macrophages at the lesion site;

wherein at least one of the first therapeutic agent or the second therapeutic agent is delivered systemically;

wherein the polymer is capable of controlled release of at least one of the first therapeutic agent or the second therapeutic agent; and

wherein first therapeutic agent and the second therapeutic are effective to promote lesion repair utilizing the device as a scaffold for tissue formation to prevent the formation of, enlargement of, and/or rupture of the aneurysm at the lesion site.

In accordance with another aspect, there is disclosed a process for preventing formation of, enlargement of, and/or rupture of an aneurysm in a subject comprising:

A process for preventing formation of, enlargement of, and/or rupture of an aneurysm in a subject comprising:

locating a device comprising a polymer at a site within a vasculature of the subject, wherein the polymer comprises a first therapeutic agent and a second therapeutic agent incorporated therein, and wherein the polymer is capable of controlled release of the first therapeutic agent and the second therapeutic agent;

wherein the first therapeutic agent comprises MCP-1 and/or osteopontin in an amount effective to promote recruitment of macrophages to the lesion site; and

wherein the second therapeutic agent comprises an inhibitor of at least one of IL-17A, CXCL1, or IL-6 effective to promote conversion of an amount of the macrophages to M2 phenotype macrophages at the site.

In certain embodiments, the device is at least partially coated with a coating comprising the polymer. In an embodiment, the coating comprises an outermost layer comprising the first therapeutic agent and an inner layer comprising the second therapeutic agent.

In accordance with yet another aspect, there is provided a device effective in treating a lesion at a lesion site in a subject comprising:

a polymer incorporating a first therapeutic agent and a second therapeutic agent therein, wherein the polymer is capable of controlled release of the first therapeutic agent and/or the second therapeutic agent;

wherein the first therapeutic agent comprises MCP-1 and/or osteopontin in an amount effective to promote recruitment of macrophages to the lesion site upon positioning of the device at the lesion site; and

wherein the second therapeutic agent comprises an inhibitor of at least one of IL-17A, CXCL1, or IL-6 effective to promote polarization of an amount of the macrophages to M2 phenotype macrophages upon positioning of the device at the lesion site.

In accordance with yet another aspect, there is disclosed a composition effective in treating a lesion at a lesion site in a subject comprising:

a first therapeutic agent, a second therapeutic agent therein, and a pharmaceutically effective carrier,

wherein the first therapeutic agent comprises MCP-1 and/or osteopontin in an amount effective to promote recruitment of macrophages to the lesion site; and

wherein the second therapeutic agent comprises an inhibitor of at least one of IL-17A, CXCL1, or IL-6 effective to promote polarization of an amount of the macrophages to M2 phenotype macrophages at the lesion site.

In still another aspect, there is disclosed a process of treating a lesion at a lesion site in a subject comprising administering an amount of MCP-1 and osteopontin to the subject effective to promote recruitment of macrophages to the lesion site, wherein the administering comprises releasing MCP-1 for a first predetermined duration followed by releasing osteopontin for a second predetermined duration.

In accordance with another aspect, there is disclosed a composition effective in treating a lesion at a lesion site in a subject comprising MCP-1, osteopontin, and a pharmaceutically effective carrier, wherein the MCP-1 and osteopontin are combined with the pharmaceutically effective carrier such that MCP-1 is released for a first predetermined duration of time and osteopontin is released for a second predetermined duration of time.

### BRIEF DESCRIPTION OF THE FIGURES

FIG. 1 shows controlled sequential release of MCP-1 and OPN from multidrug-release coils. FIG. 1A shows the results of a multi-layered bioerodible polymer-coated coils with MCP-1 in the outer layer (1st protein released), and OPN in the inner layer (2nd protein released). FIG. 1B shows MCP-1 alone-eluting coils. FIG. 1C shows OPN alone-eluting coils. FIG. 1D shows PLGA-only coils were placed in 100 μL phosphate-buffered saline (PBS) and shaken in a 37° C. incubator.

FIG. 2 provides a graph showing release of MCP-1 of coils that have been treated with polymers containing MCP-1 measured at 2 days, 4 days and 6 days.

FIG. 3 provides a graph showing prolonged release of OPN for at least 12 days in a CG910 polymer.

FIG. 4 provides a graph showing that MCP and CG910-MCP stop release of MCP after day 6.

FIG. 5 provides a graph showing that OPN/MCP continue to release OPN until at least day 12. OPN-CG910-MCP1 and OPN-CG910 (2)-MCP1 continue to release until day 18.

FIG. 6 provides a graph showing that coils coated with CG910 containing OPN/MCP continue to release both proteins for at lease 12 days.

FIG. 7 provides a graph showing that the addition of CG910-MCP1 treatment accelerates the rate at which MCP-1 stops being released, but it does not delay the rate of MCP-1 release. No dip persists until at least day 12? One dip stops at day 10. Two dips stops at day 4.

FIG. 8 provides a graph showing release of OPN in PLGA as a polymer at 2 days, 4 days and 6 days.

FIG. 9 provides a graph showing OPN release of groups 0, 1, or 2 above at 20× dilution.

FIG. 10 provides a graph showing MCP1 release at days 2, 3, 6, 8, 10, and 12 from Groups 0, 1, 2, 3, 4, and 5.

FIG. 11 provides a graph showing release of OPN and MCP-1 from coils treated with compositions described herein.

FIG. 12 provides a graph showing release of OPN and MCP-1 from coils treated with compositions described herein.

FIG. 13 illustrates an aneurysm coil that may be treated with compositions described herein to release certain agents at an aneurysm site.

FIG. 14 illustrates a stent device for stent-assisted coil employment that may be treated with compositions described herein to release certain agents at an aneurysm site.

### DETAILED DESCRIPTION

Aspects of the present invention will now be described more fully hereinafter with reference to specific embodiments. Indeed, aspects of the present invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an,” “the,” include plural referents unless the context clearly dictates otherwise.

Elsewhere, as used herein, the terms “administering” or “administration” of a therapeutic agent as described herein includes any route of introducing or delivering to a subject a compound to perform its intended function. The administering or administration can be carried out by any suitable route, including systemically, orally, intranasally, parenterally (intravenously, intramuscularly, intraperitoneally, or subcutaneously), or topically. Administering or administration includes self-administration and the administration by another.

As used herein, the terms “effective amount,” “therapeutically effective amount,” or the like refer to an amount which, when administered in a proper dosing regimen, is sufficient to enhance or improve the repair of a lesion in a subject, or hinder or prevent occurrence of lesion formation (relative to the absence of such administration). The full therapeutic effect does not necessarily occur by administration of one dose and may occur only after administration of a series of doses of the subject therapeutic agent. Thus, an effective amount or therapeutically effective amount may be administered in one or more administrations per day for successive days.

As used herein, the term “lesion” includes a region in an organ or tissue comprising an irregularity in its structure due to injury, disease, or the like. A lesion includes but is not limited to vascular lesions (e.g., an aneurysm), wounds, burns, and ulcers.

As used herein, the term “pharmaceutically acceptable carrier” refers to a carrier medium which does not interfere with the effectiveness of the biological activity of the therapeutic agents and which is not excessively toxic to the subject at the concentration at which it is administered. The term includes solvents, dispersion media, coatings, antibacterial and antifungal agents, isotonic agents, absorption delaying agents, and the like. The use of such media and agents for pharmaceutically active substances is well known in the art.

As used herein, the terms “repair” or “lesion repair” refer to any aspect of lesion repair (e.g., speed of repair, extent of repair, or the like), including the prevention of formation of a lesion from forming or a mitigating or eliminating a symptom of a lesion in a subject.

As used herein, the term “subject” refers to an animal being treated with one or more of the therapeutic agents as taught herein, including, but not limited to, simians, humans, avians, felines, canines, equines, rodents, bovines, porcines, ovines, caprines, mammalian farm animals, mammalian sport animals, and mammalian pets. A suitable subject for various embodiments can be any animal, e.g., a human, that is suspected of having, has been diagnosed as having, or is at risk of developing a lesion that can be ameliorated, treated or prevented by administration of one or more therapeutic agents as described herein.

As used herein, the term “systemic administration” refers to administration of an agent such that the agent becomes widely distributed in the body in an amount sufficient to bring about its intended biological effect, e.g., reaches its desired site of action via the vascular system of the subject. Typical systemic routes of administration include administration by (1) introducing the agent directly into the vascular system or (2) oral, pulmonary, or intramuscular administration wherein the agent is adsorbed, enters the vascular system, and is carried to the lesion site via the vascular system.

As used herein, the terms “treatment,” “treating,” or the like means that administration of the therapeutic agents described herein improves an aspect of lesion repair (e.g., speed of repair, extent of repair, or the like) and/or prevents, alleviates, ameliorates, inhibits, or mitigates the formation of a lesion in a subject to a degree relative to the absence of the administration of therapeutic agents described herein.

The present inventors have found that current therapies for the treatment, including prevention and repair, of lesions do not adequately control the presence of and timing of certain critical factors involved in inflammatory repair. For example, conventional therapies have, for example, provided a greater amount of certain macrophages at lesion sites when their presence is no longer desired, e.g., in the repair and remodeling stages of lesion healing. As such, certain macrophages may be involved in the breakdown of tissue at lesion sites when factors that promote repair of the tissue are instead preferred. Aspects of the present disclosure are thus directed to methods of treatment, devices, and compositions which better control and promote the desired macrophage phenotype present at a lesion site at desired time interval to improve the treatment of lesions, including but not limited to vascular injury, ulcers, burns, wounds, and the like. In certain embodiments, the processes, devices, and compositions may be used in vasculature generally and can be used to treat vascular injuries, including the treatment of aneurysms.

In one aspect, there is disclosed a general process of treating a lesion at a lesion site in a subject. The process includes (first) administering an amount of a first therapeutic agent comprising MCP-1 and/or osteopontin to the subject effective to promote recruitment of macrophages to the lesion site. Thereafter, the process includes administering an amount of a second therapeutic agent comprising an inhibitor of at least one of IL-17A, CXCL1, or IL-6 effective to promote conversion of an amount of the macrophages to M2 phenotype macrophages at the lesion site, wherein the M2 phenotype macrophages promote enhance lesion repair in the repair and remodeling stages thereof.

The first therapeutic agent as described herein comprises at least one of MCP-1 or osteopontin. MCP-1 (monocyte chemoattractant protein 1) (also referred to as CCL2) is a small cytokine belonging to the CC chemokine family. It is a monomeric polypeptide having a molecular weight of approximately 13-15 kDa, depending on its degree of glycosylation. MCP-1 is also anchored in the plasma membrane of endothelial cells by glycosaminoglycan side chains of proteoglycans. Further, MCP-1 is primarily secreted by monocytes, macrophages and dendritic cells. In an embodiment, when administered to a subject, MCP-1 is believed to be at least effective to promote recruitment of macrophages to a lesion site, e.g., promote a greater number of macrophages to the lesion site than without administering MCP-1.

Osteopontin (OPN) is a chemokine-like protein secreted by a variety of cell-types and is an intracellular signaling complex that regulates cell adhesion, migration, and bone regeneration. It is a negatively charged aspartic acid-rich, N-linked glycosolated phosphoprotein composed of approximately 300 amino acids residues (314 amino acid residues in humans) and has about thirty carbohydrate residues attached, which are attached to the protein during post-translational modification in the Golgi apparatus. Osteopontin is expressed in a range of immune cells, including macrophages, neutrophils, dendritic cells, T cells, and B cells, with varying kinetics. In an embodiment, when administered to a subject, osteopontin is also believed to be at least effective to promote recruitment of macrophages to a lesion site, e.g., promote a greater number of macrophages to the lesion site than without administering osteopontin.

In certain embodiments, the first therapeutic agent comprises an amount of both MCP-1 and osteopontin. In addition, in the first therapeutic agent, MCP-1 and/or osteopontin are provided in an amount effective to promote subject effective to promote recruitment of macrophages to the lesion site. In an embodiment, the macrophages comprise M1 phenotype macrophages which are believed to create a pro-inflammatory environment and remove extracellular debris by phagocytosis.

The second therapeutic agent comprises an inhibitor of one or more of IL-17A, CXCL1, or IL-6. In an embodiment, the second therapeutic agent comprises an inhibitor of both IL-17A and an inhibitor of CXCL1. In another embodiment, the second therapeutic agent comprises an inhibitor of each of IL-17A, CXCL1, and IL-6.

IL-17A is a pro-inflammatory cytokine which belongs to the IL-17 family consisting of six isoforms (IL-17A-F). IL-17A has been found to play a role in neutrophil recruitment, host defense, and immuno-inflammatory pathology. See Postepy Dermatol Alergol. 2016 August; 33(4): 247-252. The IL-17A inhibitor may comprise any suitable entity effective to inhibit activity of IL-17A in the subject. In certain embodiments, the IL-17A inhibitor may comprise an IL-17A antagonist capable of suppressing the binding of an IL-17A receptor or IL-17A, or a substance capable of inhibiting the expression of IL-17A or an IL-17A receptor in the subject. In an embodiment, the IL-17A inhibitor may include an anti-IL-17A monoclonal antibody. An anti-human IL-17A antibody to antagonize IL-17A is set forth in WO2007/117749, for example. In other embodiments, the IL-17A inhibitor may comprise a compound of inhibiting activity of IL-17A in the subject from the group consisting of brodalumab, secukinumab, ixekizumab, celastrol, or the like, and combinations thereof. It is believed that inhibition of IL-17A at the repair and remodeling stages of the lesion healing process will promote lesion healing by promoting an anti-inflammatory environment at such stages. In addition, it is believed that the administration of the IL-17A inhibitor during such stages will promote polarization of macrophages to M2 phenotype macrophages.

CXCL1 (also referred to as C-X-C motif chemokine 1) is a small cytokine belonging to the CXC chemokine family. It has been found that human aneurysms, for example, express CXCL1, whereas normal arteries do not. Hypertension, 2014 December; 64(6): 1306-1313. As with IL-17A, it is believed that inhibition of CXCL1 during the repair and remodeling stages will promote lesion healing by promoting an anti-inflammatory environment at the lesion site during such stages. In addition, it is believed that the administration of the CXCL1 inhibitor during such stages will promote polarization of macrophages to M2 phenotype macrophages. The CXCL1 inhibitor may comprise any suitable entity effective to inhibit activity of CXCL1 in the subject. In certain embodiments, the CXCL1 inhibitor may comprise an CXCL1 antagonist capable of suppressing the binding of an CXCL1 receptor or CXCL1, or a substance capable of inhibiting the expression of CXCL1 or an CXCL1 receptor in the subject. Exemplary inhibitors of CXCL1 include but are not limited to an anti-CXCL1 antibody.

IL-6 Interleukin 6 (IL-6) is an interleukin that acts as both a pro-inflammatory cytokine and an anti-inflammatory myokine. IL-6 is encoded by the IL-6 gene in humans. As with IL-17A and CXCL1, it is believed that inhibition of IL-6 at the repair and remodeling stages will promote lesion healing by promoting an anti-inflammatory environment at the lesion site during such stages. In addition, it is believed that the administration of the IL-6 inhibitor during such stages will promote polarization of macrophages to M2 phenotype macrophages during such stages. The IL-6 inhibitor may likewise comprise any suitable entity effective to inhibit activity of IL-6 in the subject. In certain embodiments, the IL-6 inhibitor may comprise an IL-6 antagonist capable of suppressing the binding of an IL-6 receptor or IL-6, or a substance capable of inhibiting the expression of IL-6 or an IL-6 receptor in the subject. Exemplary IL-6 inhibitors include but are not limited to tocilizumab, siltuximab, sarilumab, olokizumab, elsilimomab, sirukumab, clazakizumab, and the like.

Without wishing to be bound by theory, it is believed that the second therapeutic agent acts to decrease or otherwise inhibit at least IL-17A, CXCL1, and/or IL-6 to reduce the number of proinflammatory factors, including but not limited to IL-17A, CXCL1, and/or IL-6, at the lesion site. In certain embodiments, the second therapeutic agents may also decrease in number inhibit other factors which would interfere with the repair and remodeling phases of lesion healing. In addition, it is believed that the administration of the second therapeutic agent will further contribute to lesion healing by further promoting an anti-inflammatory environment via promoting polarization of macrophages to M2 macrophages via macrophage polarization. It is appreciated that the macrophages converted via macrophage polarization to M2 phenotype macrophages will be macrophages other than the M2 phenotype macrophages and, in certain embodiments, include at least M1 phenotype macrophages.

It is also appreciated that any suitable methods and/or materials may be utilized to confirm the polarization of macrophages to anti-inflammatory M2 phenotype macrophages. In an embodiment, suitable biomarker(s) may be utilized for the detection of M2 phenotype macrophages (or other phenotypes) in order to confirm conversion to the M2 phenotype. In a particular embodiment, for example, inducible nitric oxide synthase (iNOS) may be utilized as a marker for M1 phenotype macrophages, if needed, and a CD206 marker may be utilized as a marker for the M2 phenotype as set forth in J. Neurointerv Surg., 2018 March; 10(3): 301-305.

The first and second therapeutic agents may be provided in any suitable form for administration to the subject. In an embodiment, the first and second therapeutic agents are provided within a suitable pharmaceutically acceptable carrier. The pharmaceutically acceptable carrier may take a wide variety of forms depending upon the desired route of administration (e.g., for systemic administration as described herein). For liquid oral preparations (e.g., suspensions, elixirs and solutions), suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and/or other ingredients may be added to increase solubility or preservation. Injectable suspensions or solutions may also be prepared utilizing aqueous carriers along with appropriate additives.

In an aspect, the first and/or second therapeutic agent may be incorporated within the pharmaceutically acceptable carrier for controlled release of the first and/or second therapeutic agent over a predetermined duration. In certain embodiments, both the first and/or second therapeutic agents are incorporated within the pharmaceutically acceptable carrier for sequential (controlled) release of the first and second therapeutic agents. For example, the first therapeutic agent may be released over a first predetermined time duration followed by controlled release of the second therapeutic agent over a second predetermined time duration. In an embodiment, the first and second predetermined time duration overlap, but it is understood that the present disclosure is not so limited.

In an embodiment, the pharmaceutically acceptable carrier comprises a polymer. Thus, in an embodiment, the first and/or second therapeutic agents may be provided within a polymer capable of controlled release of the first and/or second therapeutic agents for administration to the subject or for application to a lesion repair device such as an aneurysm coil and/or stent. Suitable polymers for controlled release of the first and/or second therapeutic agents are disclosed in WO 2008/115978, the entirety of which is hereby incorporated by reference herein. To provide an introduction for the same, the polymer may comprise any type of polymer capable of use in the human body (biocompatible polymers) and capable of maintaining and controlled release of a therapeutic agent as described herein to a lesion site for treatment of a subject. In an embodiment, the polymer comprises a biodegradable polymer.

In a particular embodiment, the polymer comprises poly(lactide-co-glycolide) (PLGA). PLGA is a biodegradable polymer demonstrated to promote a consistent cellular reaction both in vitro and in vivo by slow acid release during its degradation, and itself may locally serve as a potent chemokine for signaling of tissue macrophages and fibroblasts. The PLGA utilized in treatment may have any suitable ratio of glycolic acid and lactic acid, such as a lactic acid to glycolic acid monomeric ratio of from 80:20 to 20:80, 75:25 to 25:75, 60:40 to 40:60, or 50:50. In other embodiments, the polymer may comprise polytrimethylene carbonate (pTMC). Additives, such as magnesium hydroxide, may be added to adjust the degradation rate or the local pH of polymer composition.

In still other embodiments, the polymer may comprise a non-degradable polymer. Exemplary non-degradable polymers include polyethylene terephthalate (PET), polyethylene naphthalate (PEN), polypropylene (PP), and polytetrafluoroethylene (PTFE). When non-biodegradable polymers are used, such polymers may be modified to comprise surface immobilized or controlled release biochemical attractors, e.g., such as by being constructed with porosity. Likewise, a variety of porous structures, such as polyvinyl acetate copolymers, can be used as controlled release systems. Porous non-degradable or degradable polymers may further offer staged controlled release by precipitation of immobilizing agents in layers within pores thereof. In an embodiment, polymers for use in the present disclosure can further be surface modified or otherwise formed to release antithrombotic agents to assist their function. Still further, hydrogel polymers, e.g., poly-hydroxy ethylmethacrylate (pHEMA) or polyelectrolyte complexes, could also be used according to the disclosure. Examples of specific further polymers that could be used include fibrin polymer and collagen polymers, as well as combinations of degradable and non-degradable polymers. Still other polymer compositions useful in the present disclosure include oxidized cellulose, crosslinked protein (e.g., casein or albumin), alginate, and polyethylene oxide (PEO) hydrogels.

In other embodiments, the polymer used to administer to a subject or apply to a lesion repair device comprises CG910 or CG950 or a combination thereof.

In certain embodiments, the first and second therapeutic agents are configured for sequential release to the subject. For example, in certain embodiments, the first therapeutic agent may be incorporated within a first pharmaceutically acceptable carrier and the second therapeutic agent within a second pharmaceutically acceptable carrier, wherein the first pharmaceutically effective carrier provides a more immediate or relatively faster delivery to the subject and the second pharmaceutically acceptable carrier provides a relatively slower or more sustained delivery of the second therapeutic agent to the substrate. In some embodiments, a release rate can be quantified as a number of days needed to release a majority of or substantially all of the respective therapeutic agent from the pharmaceutically acceptable carrier. In still other embodiments, the first and second therapeutic may be configured for sequential release from the same material, e.g., a polymer coating, by incorporating the first and second therapeutic agents in distinct layers.

In certain embodiments, the sequential release of the first and second therapeutic agents can be accomplished by the polymers described herein, and further including the use of microspheres. In an embodiment, the microspheres comprise polymeric microspheres, e.g., a PLGA polymeric microspheres. A variety of polymeric delivery systems are known in the art and may be utilized herein. Varde and Pack 2004 Expert Opin Biol Ther 4, 35-51. The polymeric microspheres may have a size range of 0.1 μm to 500 μm. In addition, the release rate of the microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG, sodium hyaluronate, diketopiperazine derivatives, calcium phosphate-PEG particles, and/or oligosaccharide derivative DPPG.

The polymer composition comprising a polymer and the first and/or second therapeutic agents as described herein may be utilized for lesion repair or may be incorporated in, or included with, another treatment, such as a medical device. In a particular aspect, the present disclosure includes devices useful in vascular lesion repair. For example, in some embodiments, the device may comprise a stent (e.g., a coronary stent, peripheral stent, carotid stent, intracranial stent, or a stent graft) or an aneurysm coil. In an embodiment, the polymer at least partially coats a pre-made stent or aneurysm coil.

In further embodiments, the device can partially or totally formed from the polymer composition. For example, the polymer can be used to form a device, such as a scaffold, to be placed within a damaged vessel or vasculature. In specific embodiments, the polymer could be used by itself to form a stent or a coil which is delivered to the subject endovascularly. In other embodiments, the device may also be delivered by one or more surgical techniques.

The devices and processes described herein are suitable for lesion repair, including treatment in a subject of suspected lesions or to delay or otherwise prevent a lesion from forming. In an embodiment, the lesion comprises a member from the group consisting of a vascular injury, ulcer, burn, and a wound, although it is understood that the present disclosure is not so limited. In particular embodiment, the lesion is a vascular injury. In an embodiment, the vascular injury is an aneurysm and the administering of the first and/or second therapeutic agents (or composition or device including the same) is effective to prevent formation of, enlargement of, and/or rupture of an aneurysm in a subject.

In certain embodiments, the first and second therapeutic agents are both administered to the subject. The following paragraphs will further discuss administration of the first and second therapeutic agents, which necessarily includes administration of the first therapeutic agent. In an embodiment, the first therapeutic agent is administered to the subject prior to the second therapeutic agent, and the second therapeutic agent is administered to the subject after a predetermined duration from a start of the administering of the first therapeutic agent. In this way, the first therapeutic agent promotes macrophage recruitment to the lesion site in the subject and, after a suitable time period (the predetermined duration), the second therapeutic agent is administered to then promote polarization of an amount of the macrophages at the lesion site to M2 phenotype macrophages. In this way, an anti-inflammatory response and environment is further enhanced in the subject at the lesion site when needed, thereby enhancing the subject's own natural lesion healing response. In an embodiment, the predetermined duration from time of first administration of the first therapeutic agent to first administration of the second therapeutic agent is at least one day, and in a particular embodiment, is from 2 days to 2 weeks.

Further, the first therapeutic agent and second therapeutic agent may be administered over a predetermined time period to provide their respective desired effects. In an embodiment, the first therapeutic agent is administered to the subject for a first predetermined duration, and wherein the first predetermined duration comprises from 1 day to 10 weeks, and in a particular embodiment, from 5 days to 5 weeks. In other embodiments, the first predetermined duration is from 1 to 14 days. Additionally, in an embodiment, the second therapeutic agent is administered to the subject for a second predetermined duration, and wherein the second predetermined duration comprises from 1 day to 10 weeks, and in particular embodiment from 5 days to 5 weeks. In other embodiments, the second predetermined duration is from 1 to 14 days.

As noted above, the first and second therapeutic agents may be administered to the subject by any suitable method of administration to bring about their intended effect. In an embodiment, the first therapeutic agent and/or the second therapeutic agent are incorporated within a pharmaceutically effective carrier, e.g., a polymer as described herein, which is located at the lesion site, and which is suitable for controlled release of the first and/or second therapeutic agents to the lesion site. In an embodiment, the polymer is provided as a coating of or as a part of device located at the lesion site. In an embodiment, the device comprises a stent or aneurysm coil. Such embodiments are thus suitable for the treatment of vascular injury, for example. In a particular embodiment, the first and/or second therapeutic agents are incorporated within an aneurysm coil which located at the lesion (e.g., aneurysm) site for controlled release of the first and/or second therapeutic agents therefrom. In other embodiments, the first and/or second therapeutic agents may be incorporated within a wound dressing or the like for direct contact with a lesion in need of healing.

In still other embodiments, the first and/or second therapeutic agents may be administered via systemic administration to the subject. The systemic administration may include introduction of the first and/or second therapeutic agents directly into the vascular system or any other method of administration (e.g., oral, pulmonary, or intramuscular administration) wherein the first and/or second therapeutic agents are adsorbed, enter the vascular system, and are carried to the lesion site via the vascular system. In an embodiment, the systemic administration is done via oral or intravenous administration.

In particular embodiments for treating an aneurysm in a subject, at least one of the first therapeutic agent or the second therapeutic agent is delivered systemically to the subject while an aneurysm coil is located at the lesion site. Accordingly, the aneurysm coil may include neither of the first and second therapeutic agents, or may include the therapeutic agent which is not delivered systemically. In a particular embodiment, both the first and second therapeutic agents are delivered systemically and an aneurysm coil (which does not include the first or second therapeutic agents) is located within the vasculature of the subject. In another embodiment, the aneurysm coil has incorporated therein one or both of the first therapeutic agent and the second therapeutic agent. In particular embodiments, the aneurysm coil comprises the first therapeutic agent, wherein the first therapeutic agent comprises MCP-1 and osteopontin. The aneurysm coil may include any suitable coil as is known in the art, such as platinum-based aneurysm coils.

In another aspect, there is thus disclosed a process for preventing formation of, enlargement of, and/or rupture of an aneurysm in a subject comprising: locating a device comprising a polymer capable of controlled release of the first and/or second therapeutic agents at a lesion site within a vasculature of the subject; and administering to the subject an amount of a first therapeutic agent and second therapeutic agent as described herein, wherein at least one of the first therapeutic agent and the second therapeutic agent is delivered systemically; and wherein first therapeutic agent and the second therapeutic are effective to promote lesion repair via utilizing the device as a scaffold for tissue formation to prevent the formation of, enlargement of, and/or rupture of the aneurysm at the lesion site.

As discussed previously, in certain embodiments, the first therapeutic agent is administered to the subject prior to the second therapeutic agent, and thereafter the second therapeutic agent is administered to the subject after a predetermined duration from a start of the administering of the first therapeutic agent. It is appreciated that selection of the route of administration may assist in allowing the predetermined duration to pass between first administration of the first therapeutic agent and the administration of the second therapeutic agent.

In still other embodiments, a device which incorporates the first and second therapeutic agents is located at the lesion site. In particular embodiments, the device comprises an aneurysm coil comprising the polymer which incorporates the first and second therapeutic agents therein and is capable of controlled release of the first and/or second therapeutic agents. Accordingly, aspects of the present disclosure also disclose a process for preventing formation of, enlargement of, and/or rupture of an aneurysm in a subject comprising locating a device comprising a polymer at a site within a vasculature of the subject, wherein the polymer capable of controlled release of the first and/or second therapeutic agents comprises a first therapeutic agent and/or a second therapeutic agent incorporated therein.

The device may comprise any suitable structure which allows the first and/or second therapeutic agents to be released from the polymer. In an embodiment, for example, the device is at least partially coated with a coating comprising a polymer as set forth herein. In such case, the coating may comprise an outermost layer comprising the first therapeutic agent and an inner layer comprising the second therapeutic agent. In this way, over a predetermined period of time, the polymer allows staged controlled release of the first therapeutic agent to the lesion site and thereafter allows release of the second therapeutic agent. It is appreciated that one skilled in the art would be able to determine the necessary thicknesses of the layer(s) to release the therapeutic agents to bring about their intended result. In other embodiments, the polymer itself forms at least a portion of the device. In still other embodiments, the first therapeutic agent and/or the second therapeutic agent may be administered to the subject via controlled release such that the components within the first and/or second therapeutic agent are delivered in stages (sequential release). For example, in an embodiment, there is disclosed a process of treating a lesion at a lesion site in a subject comprising administering an amount of MCP-1 and osteopontin (first therapeutic agent) to the subject effective to promote recruitment of macrophages to the lesion site, wherein the administering comprises releasing MCP-1 for a first predetermined duration followed by releasing osteopontin for a second predetermined duration. The administration of the MCP-1 and osteopontin may take place by any suitable method, composition, or device as described herein. In certain embodiments, the predetermined duration is for 1-14 days. Further, in embodiments, the administration of the MCP-1 and osteopontin overlap; however, it is understood that the present disclosure is not so limited. Still further, in certain embodiments, the process further comprises administering an amount of a second therapeutic agent comprising an inhibitor of at least one of IL-17A, CXCL1, or IL-6 effective to promote polarization of an amount of the macrophages to M2 phenotype macrophages at the lesion site.

In yet another aspect, there is disclosed a composition effective in treating a lesion at a lesion site in a subject comprising MCP-1, osteopontin, and a pharmaceutically effective carrier, wherein the MCP-1 and osteopontin are combined with the pharmaceutically effective carrier such that MCP-1 is released for a first predetermined duration of time and osteopontin is released for a second predetermined duration of time. In an embodiment, the pharmaceutically effective carrier may comprise a polymer as described herein capable of controlled release of the MCP-1 and osteopontin. In certain embodiments, the composition forms all of or part of a device which is administered to the subject, such as a stent or coil. In still other embodiments, the composition further comprises a second therapeutic agent comprising an inhibitor of at least one of IL-17A, CXCL1, or IL-6 as described herein.

The above disclosure discloses systems, devices, compositions, and processes capable of accelerating lesion repair in a subject by initially promoting macrophage recruitment to the lesion site and thereafter promoting an anti-inflammatory environment for tissue repair and remodeling by polarization of the recruited macrophages to M2 phenotype macrophages. Aspects of the present disclosure will now be described with specific reference to examples. The following examples are not intended to be limiting of the disclosure and are rather provided as exemplary embodiments.

### DEVICE EXAMPLES

As noted above, the disclosure provide endovascular devices that assist in treating vascular lesions such as aneurysms. A brain aneurysm is a swollen blood vessel in a person's brain that can grow and rupture. Before an aneurysm ruptures, the person may suffer severe headaches, nausea, vision impairment, vomiting, and loss of consciousness. Ruptured aneurysms are treated to prevent re-bleeding, and clipping is one such treatment. In the clipping treatment, part of the skull bone is temporarily removed and the base of the aneurysm is clipped to stop bleeding. However, clipping is invasive, difficult to perform, and brings a risk of infection.

A less invasive option is vascular embolization, in which implants such as metal coils are placed in the aneurysm to create blood stasis and promote blood clotting, which physically blocks the flow of blood into the aneurysm. In coil embolization, a microcatheter is used to guide the embolization coil to the aneurysm under x-ray or digital subtraction angiography (DSA) guidance. Typically, a pusher wire attached to the delivery device then delivers the coil into the aneurysm.

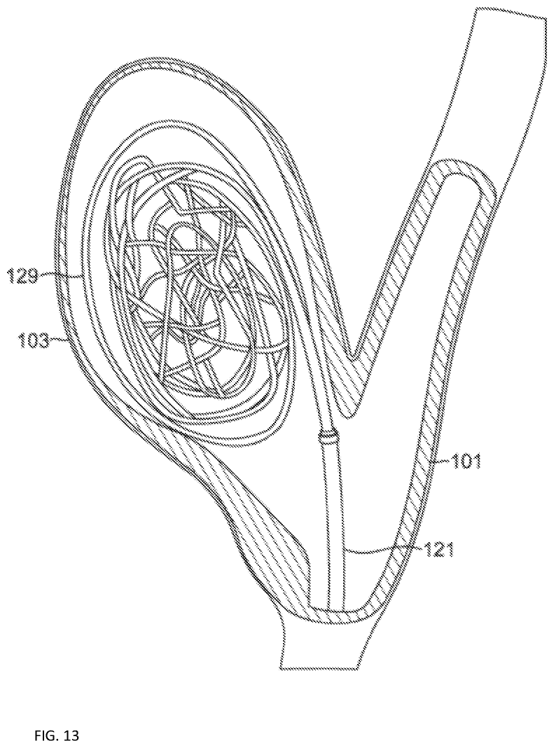

FIG. 13 illustrates an aneurysm treatment device that may be treated with compositions described herein to release certain agents at an aneurysm site. The device includes a delivery catheter 121 that is used to deliver one or a set of coils 129 to the lesion site 103 of vessel 101. As noted above, the coils 129 may be associated with at least one coating which incorporates one or more of first and second therapeutic agents as discussed above. Briefly, first therapeutic agent comprises monocyte chemotactic protein (MCP-1) and/or osteopontin. The second therapeutic agent to the subject comprising an inhibitor of at least one of IL-17A, CXCL1, or IL-6. The polymer is capable of controlled release of the first and/or second therapeutic agents. In this way, an anti-inflammatory environment is further promoted at the lesion site, particularly for the repair and remodeling stages of lesion repair at the appropriate point in time.

The catheter 121 with one or more coils 129 is inserted into an artery and advanced to the abnormal blood vessel 101 harboring the aneurysm. Once properly positioned, the coil 129 is released into position within the vessel 101. The coils are typically made of a metal (e.g. platinum). Coil 129 remains firmly in place by the foldability of the material. As a result of stasis and release of the first and second therapeutic agents, the blood will clot and form around the coil 129, completely obstructing the abnormal blood flow from the artery. Eventually a scar will form as a result of smooth muscles cells depositing at the aneurysm neck, creating a permanent seal. The polymer coating provides improved and more efficient repair of the aneurysm lesion.

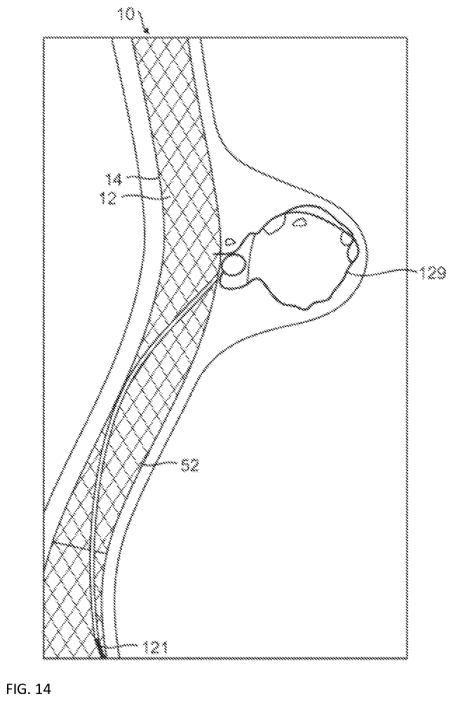

FIG. 14 depicts use of a stent device 10 for stent-assisted coil employment. A stent device 10 may include a body portion made from conventional materials such as a metal, or a metal alloy known in the art of stent-assisted coil embolization. For example, contemplated body portions may comprise stainless steel, platinum, titanium, nitinol, etc. The stent diameter is typically between 1.5 and 6.0 mm, the stent wall thickness is typically between 30 and 100 microns and the stent surface is coated via vapor deposition with a thin 5 to 30 micron layer of tantalum.

The material of stent device 10 may have a surface 12 that is coated with at least one polymer coating 14 that comprises a first therapeutic agent and a second therapeutic agent that facilitates better repair of the aneurysm lesion. The stent device may have open cells 52 or other configurations known in the art. Also shown is a catheter 121 for deploying a coil 129.

### EXPERIMENTAL

Aspects of the present disclosure will now be described with specific reference to examples. The following examples are not intended to be limiting of the disclosure and are rather provided as exemplary embodiments.

### Example 1: Multilayered Multifactor Bioerodible-System Polymer-Based Coils

Multilayered bioerodible-system polymer-based coils are created that control release of MCP-1 and OPN. The multifactor-eluting coils are created by dipping standard platinum aneurysm coils into an aqueous protein suspension consisting of 10 mg/mL of the inner layer factor (the factor that is to be released in the 2nd phase) in 50:50 poly-DL-lactic glycolic acid (PLGA; Sigma-Aldrich) and dichloromethane anhydrous (Sigma-Aldrich) neutralized to a pH of 7.0 with Mg(OH)₂ and dried for 24 hours at 4° C. Thereafter, the coils are then dipped into a 2nd aqueous protein suspension consisting of 10 mg/mL of the outer layer factor (the factor that is to be released in the 1st phase) in 50:50 poly-DL-lactic glycolic acid (PLGA; Sigma-Aldrich) and dichloromethane anhydrous (Sigma-Aldrich) neutralized to a pH of 7.0 with Mg(OH)₂ and dried for another 24 hours at 4° C. Control single-factor eluting coils consist of a single layer by dipping standard platinum aneurysm coils into an aqueous suspension of PBS in 50:50 PLGA and dichloromethane anhydrous with 10 mg/mL of MCP-1 or OPN. Control PLGA-only coils are created by dipping standard platinum aneurysm coils into an aqueous suspension of PBS in 50:50 PLGA and dichloromethane anhydrous without protein. Table 1 provides information of the release properties of the created coils.

TABLE 1

| Multilayered | Outer Layer | Inner Layer |
|---|---|---|
| Coil | (released in 1^{st} phase) | (released in 2^{nd} phase) |
| 1 | MCP-1 for 11 days | OPN for 10 days |
| 2 | MCP-1 for 14 days | OPN for 7 days |
| 3 | MCP-1 for 7 days | OPN for 14 days |
| 4 | OPN for 11 days | MCP-1 for 10 days |
| 5 | OPN for 14 days | MCP-1 for 7 days |
| 6 | OPN for 7 days | MCP-1 for 14 days |
| 7 | MCP-1 and OPN | |
| | concurrently for 21 | |
| | days | |
| 8 | MCP-1 alone for 21 | |
| | days (control) | |
| 9 | OPN alone for 21 | |
| | days (control) | |
| 10 | PLGA alone (control) | |

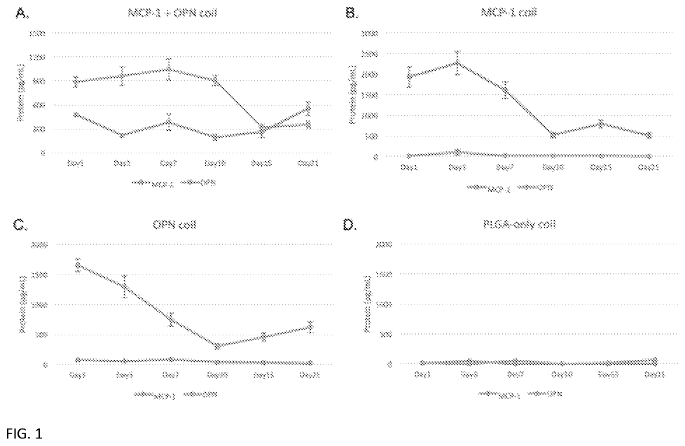

FIG. 1 shows controlled sequential release of MCP-1 and OPN from multidrug-release coils. FIG. 1A shows the results of a multi-layered bioerodible polymer-coated coils with MCP-1 in the outer layer (1st protein released), and OPN in the inner layer (2nd protein released). FIG. 1B shows MCP-1 alone-eluting coils. FIG. 1C shows OPN alone-eluting coils. FIG. 1D shows PLGA-only coils were placed in 100 μL phosphate-buffered saline (PBS) and shaken in a 37° C. incubator. PBS was collected followed by adding 100 μL of fresh PBS collected at 24 hours, 3 days, 7 days, 10 days, 15 days, and 21 days. Enzyme-linked immunosorbent assay (ELISA) was performed for each sample and levels of MCP-1 and OPN were quantified. MCP-1+OPN combination coil released MCP-1 early and OPN late as intended.

### Example 2: Protocol Summary for Coil Creation

The coils are created by dipping a standard endovascular platinum aneurysm coils into an aqueous protein suspension (100 uL) consisting of MCP-1 (2 mg/mL) or OPN (0.1 mg/mL) (R&D systems), in 1 mL of polymer solution, which consists of 50 mg of 50:50 PLGA, CG910 or CG950 and 1 mL of dichloromethane anhydrous (Sigma-Aldrich) neutralized to a pH of 7.0 with 30 mg of Mg(OH)₂, and then drying for 24 hours at 4° C.

### Example 3: Protein Release of Coils Coated with PLGA, CG910 and CG950

### Protocol Summary

1) Individual coils are placed in a 96 well plate

2) 100 uL of PBS is placed into each individual well

3) Coils are placed on an orbital shaker in the 37 C incubator

4) 80 uL of the PBS solution bathing the coils are removed at 48 hour intervals and stored in −20 c freezer

5) 80 uL of fresh PBS are added to each well after PBS has been removed

6) amount of released protein are measured using ELISA kit (Raybiotech inc)

Coatings designated as MCP-1 or OPN mean the designated protein that is combined with the designated polymer. Designations such as “OPN-CG910-MCP1” represents three layers of polymer with or without protein, e.g., OPN with polymer (CG910)+polymer (CG910) only (no protein)+MCP-1 with polymer (CG910). Further, the designation “OPN-CG910-MCP” represents a coil that is dipped in OPN with CG910 (3 times or otherwise indicated). CG910 one time (or otherwise indicated), and then MCP-1 with CG910 (three times or otherwise indicated) to create the coil. For the studies shown in “OPN-CG910 (2)-MCP” is dipped coils in OPN with CG910 three times, dipping CG910 twice and then MCP-1 with CG910 three times to create the coil. Typically, a polymer-only layer is put between the protein layers. Reference to no dip, 1 dip or 2 dips etc. refer to the number of polymer (no protein) dips between the protein/polymer dips. Provided below are the specific groups referenced in the figures that specify the number of dips of each layer type.

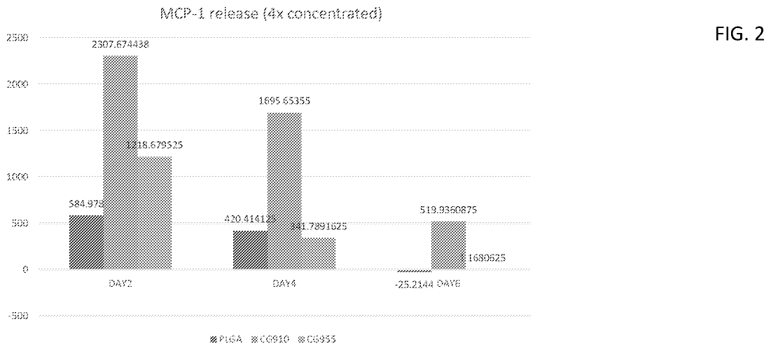

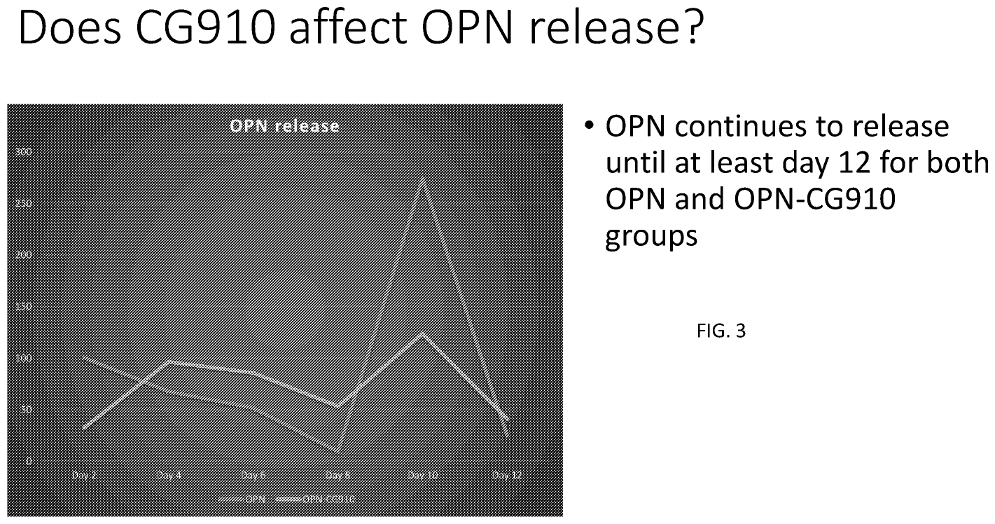

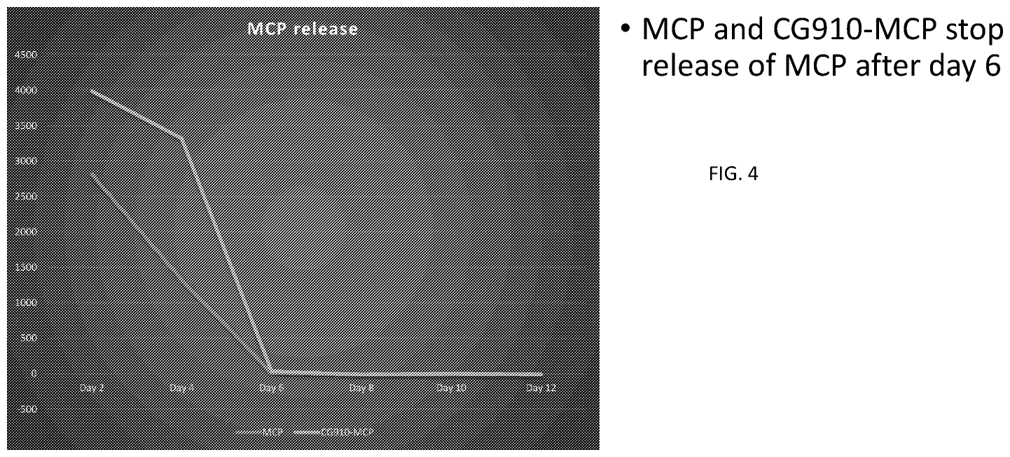

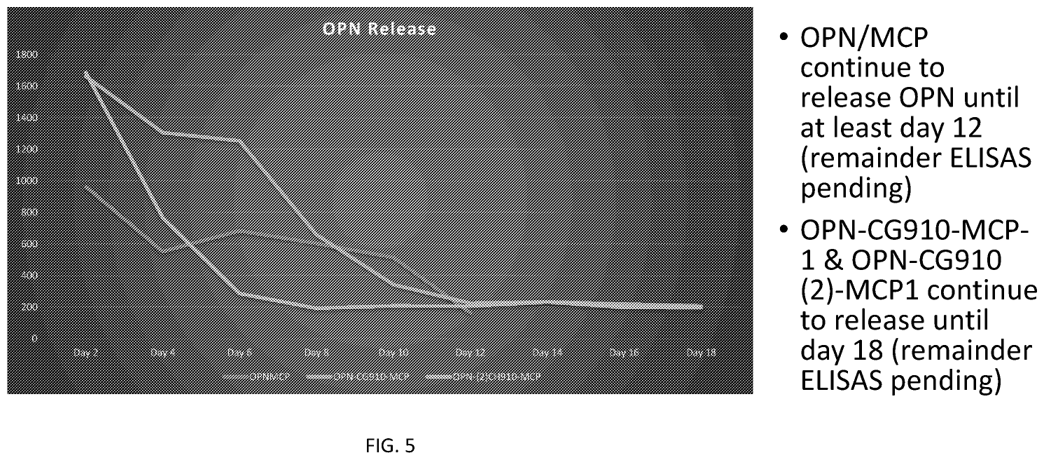

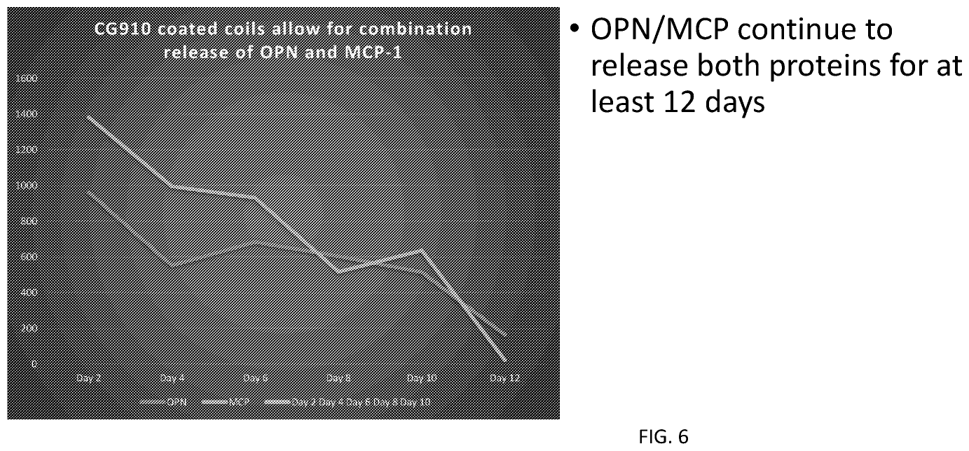

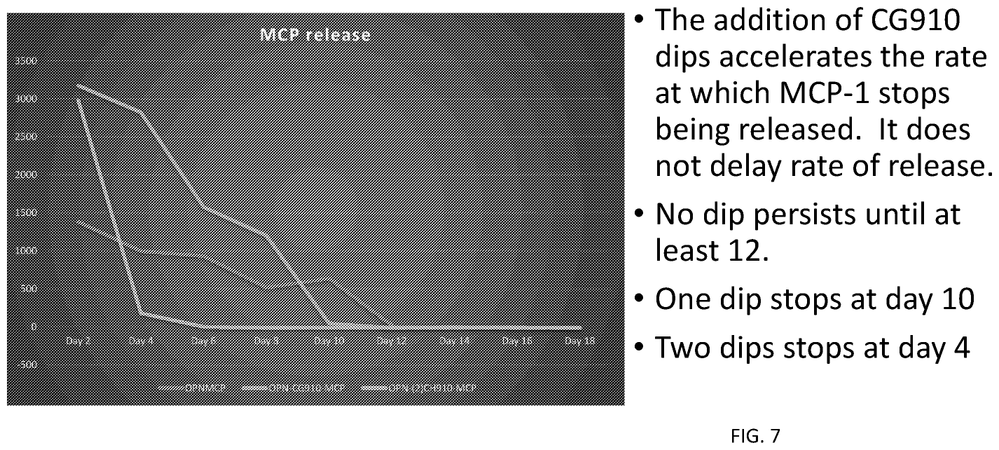

FIG. 2 provides a graph showing release of MCP-1 of coils that have been treated with polymers containing MCP-1 measured at 2 days, 4 days and 6 days. The graph shows that the CG910 releases MCP-1 at a slower and longer rate. FIG. 3 provides a graph showing prolonged release of OPN for at least 12 days in a CG910 polymer. FIG. 4 shows that MCP and CG910-MCP stop release of MCP after day 6. FIG. 5 shows that OPN/MCP continue to release OPN until at least day 12. OPN-CG910-MCP1 and OPN-CG910 (2)-MCP1 continue to release until day 18. FIG. 6 shows that coils coated with CG910 containing OPN/MCP continue to release both proteins for at lease 12 days. FIG. 7 shows that the addition of CG910-MCP1 treatment accelerates the rate at which MCP-1 stops being released, but it does not delay the rate of MCP-1 release. No dip persists until at least day 12. One dip stops at day 10. Two dips stops at day 4.

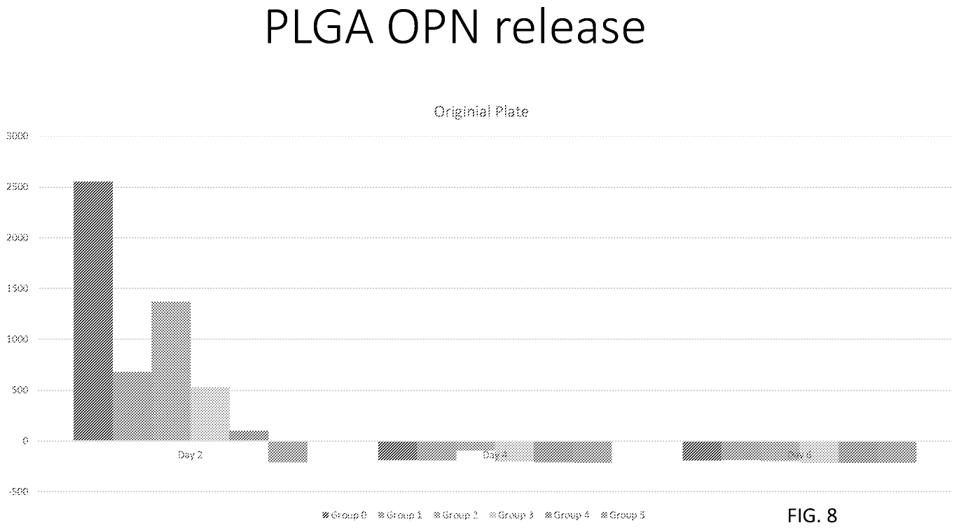

For FIG. 8 shows release of OPN in PLGA as a polymer at 2 days, 4 days and 6 days. The Groups 0, 1, 2, 3, 4, and 5 related to coils treated as follows:
Group 0: OPN dipped 3× followed by MCP dipped 3×
Group 1: OPN dipped 3× followed by PBS/buffer dipped once followed by MCP dipped 3×
Group 2: OPN dipped 3× followed by PBS/buffer dipped twice followed by MCP dipped 3×
Group 3: OPN dipped 3× followed by PBS/buffer dipped 3× followed by MCP dipped 3×
Group 4: OPN dipped 3× followed by PBS/buffer dipped 4× followed by MCP dipped 3×
Group 5: OPN dipped 3× followed by PBS/buffer dipped 5× followed by MCP dipped 3×

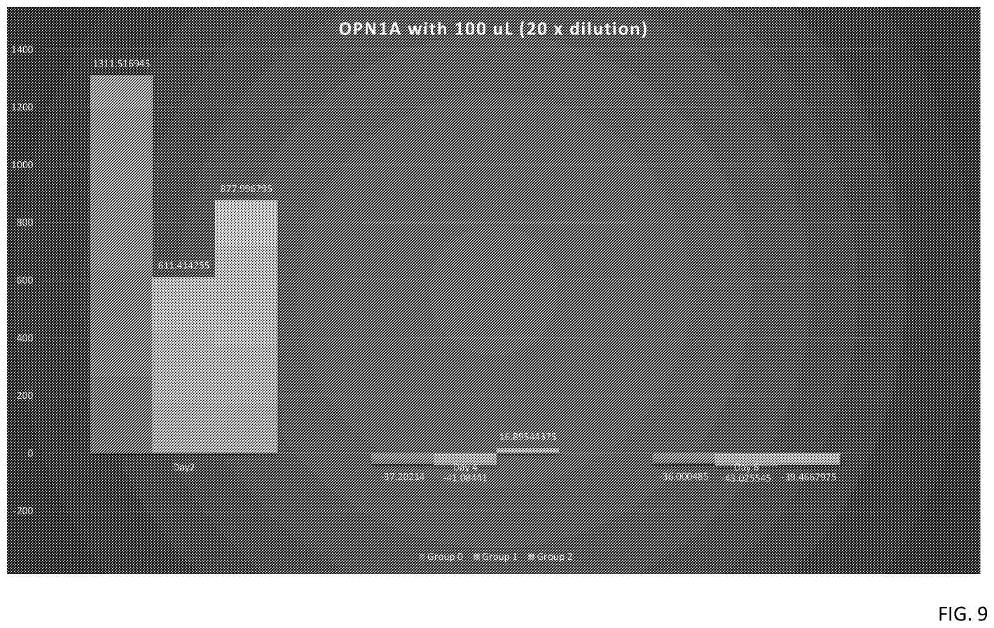

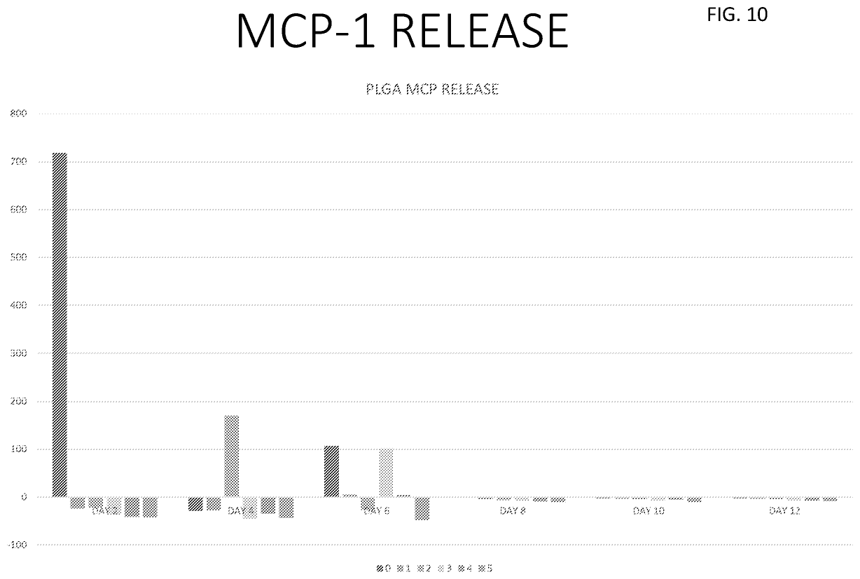

FIG. 9 shows OPN release of groups 0, 1, or 2 above at 20× dilution. FIG. 10 shows MCP1 release at days 2, 3, 6, 8, 10, and 12 from Groups 0, 1, 2, 3, 4, and 5.

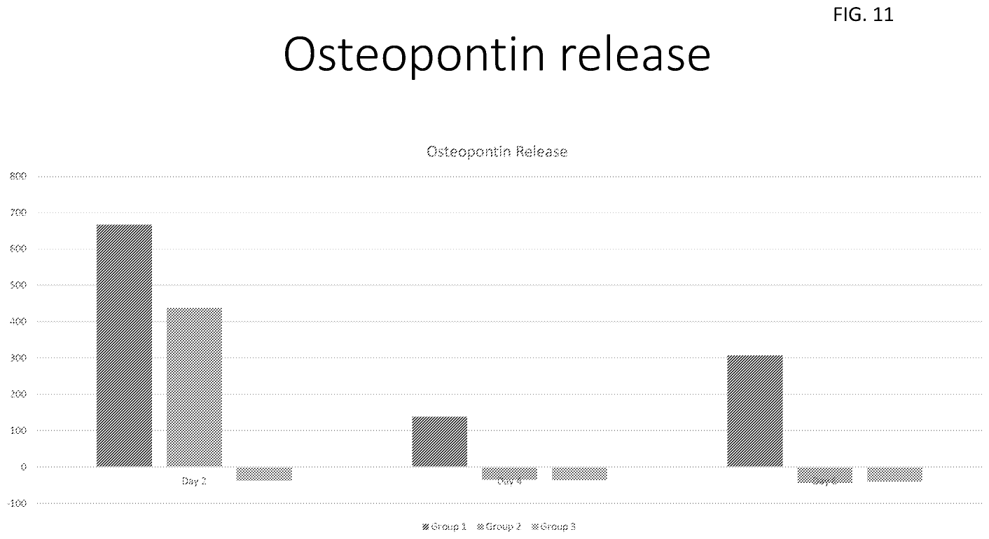

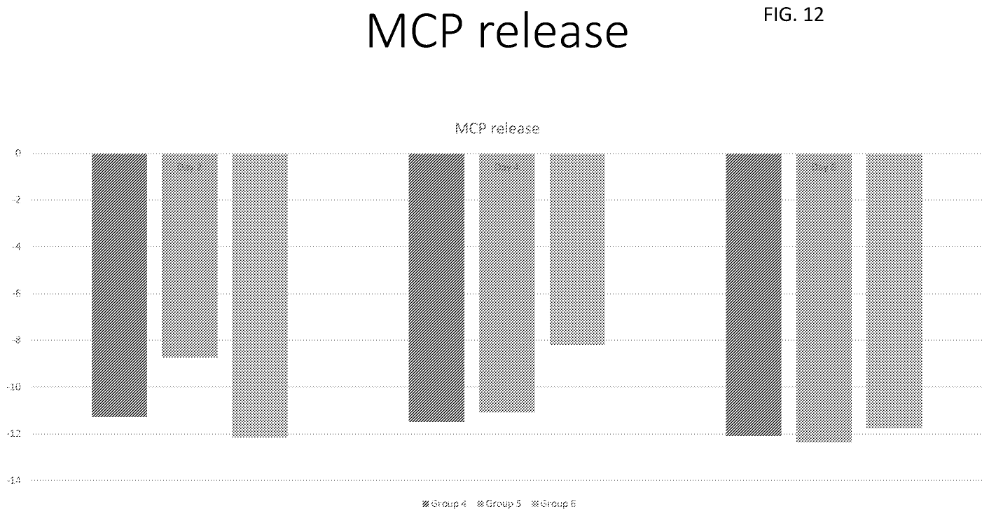

FIGS. 11 and 12 show release of OPN and MCP-1 from coils treated as follows:

Group 1: OPN dipped six times

Group 2: OPN dipped 3×

Group 3: OPN dipped 3×, f/b PLGA dipped 3×

Group 4: MCP-1 dipped 6×

Group 5: MCP-1 dipped 3×

Group 6: PLGA dipped 3× f/b MCP-1 dipped 3×

FIG. 11 shows the release of OPN at days 2, 4, and 6. FIG. 12 show MCP-1 release at days 2, 4 and 6.

All publications, patents, and patent applications referred to herein are incorporated by reference in their entirety to the same extent as if each individual publication, patent, or patent application was specifically and individually indicated to be incorporated by reference in its entirety. In the event of inconsistent usages between this document and those documents so incorporated by reference, the usage in the incorporated reference(s) should be considered supplementary to that of this document; for irreconcilable inconsistencies, the usage in this document controls.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A process of treating a lesion at a lesion site in a subject comprising:
administering an amount of a first therapeutic agent comprising MCP-1 and/or osteopontin to the subject effective to promote recruitment of macrophages to the lesion site; and
administering an amount of a second therapeutic agent comprising an inhibitor of at least one of IL-17A, CXCL1, or IL-6 effective to promote polarization of an amount of the macrophages to M2 macrophages at the lesion site.

2. The process of claim 1, wherein the first therapeutic agent is administered to the subject prior to the second therapeutic agent, and wherein the second therapeutic agent is administered to the subject after a predetermined duration from a start of the administering of the first therapeutic agent.

3. The process of claim 2, wherein predetermined duration is at least one day.

4. The process of claim 1, wherein the predetermined duration is from 2 days to 2 weeks.

5. The process of claim 1, wherein the first therapeutic agent is administered to the subject for a first duration of time, and wherein the first duration of time comprises from 1 day to 10 weeks.

6. The process of claim 1, wherein the second therapeutic agent is administered to the subject for a second duration of time, and wherein the second duration comprises from 1 day to 10 weeks.

7. The process of any of claims 1-6, wherein the lesion comprises a member from the group consisting of a vascular injury, ulcer, burn, and a wound.

8. The process of any of claims 1-6 wherein the administering of the first and second therapeutic agents prevents formation of, enlargement of, and/or rupture of an aneurysm in the subject.

9. The process of any of claims 1-8, wherein at least one of the first therapeutic agent or the second therapeutic agent is incorporated within a polymer which is located at the lesion site, and wherein the polymer is capable of controlled release of the first therapeutic agent and/or the second therapeutic agent.

10. The process of claim 9, wherein the first therapeutic agent and the second therapeutic are incorporated within the polymer.

11. The process of claim 9, wherein the polymer comprises at least one of poly(lactide-co-glycolide) (PLGA), CG910, CG950 or polytrimethylene carbonate (pTMC).

12. The process of claim 9, wherein the administering of the first or second therapeutic agent is done by locating a device comprising the polymer having at least one of the first therapeutic agent and the second therapeutic agent at the lesion site.

13. The process of claim 12, wherein the device is selected from the group consisting of a stent and an aneurysm coil.

14. The process of claim 1, wherein at least one of the first therapeutic agent or the second therapeutic agent is delivered systemically to the subject while an aneurysm coil is located at the lesion site.

15. The process of claim 1, wherein the systemic administration is done via oral or intravenous administration.

16. The process of claim 1, wherein the first therapeutic agent comprises both MCP-1 and osteopontin.

17. The process of claim 1, wherein the second therapeutic agent comprises an inhibitor of IL-17A and an inhibitor of CXCL1.

18. The process of claim 1, wherein the second therapeutic agent comprises an inhibitor of each of IL-17A, CXCL1, and IL-6.

19. A process for preventing formation of, enlargement of, and/or rupture of an aneurysm in a subject comprising:
locating a device comprising a polymer at a lesion site within a vasculature of the subject;
administering to the subject an amount of a first therapeutic agent comprising MCP-1 and/or osteopontin effective to promote recruitment of macrophages to the lesion site and a second therapeutic agent comprising an inhibitor of at least one of IL-17A, CXCL1, or IL-6 effective to promote polarization of an amount of the macrophages to M2 phenotype macrophages at the lesion site;
wherein at least one of the first therapeutic agent or the second therapeutic agent is delivered systemically;
wherein the polymer is capable of controlled release of at least one of the first therapeutic agent or the second therapeutic agent; and
wherein first therapeutic agent and the second therapeutic are effective to promote lesion repair utilizing the device as a scaffold for tissue formation to prevent the formation of, enlargement of, and/or rupture of the aneurysm at the lesion site.

20. The process of claim 19, wherein the device is selected from the group consisting of a stent and an aneurysm coil.

21. The process of claim 19 or 20, wherein both the first therapeutic agent and the second therapeutic agent are delivered systemically.

22. The process of any of claims 19-21, wherein the systemic administration is done by oral or intravenous administration.

23. The process of any of claims 19-22, wherein the first therapeutic agent comprises both MCP-1 and osteopontin.

24. The process of any of claims 19-23, wherein the second therapeutic agent comprises an inhibitor of IL-17A and an inhibitor of CXCL1.

25. The process of any of claims 19-24, wherein the second therapeutic agent comprises an inhibitor of each of IL-17A, CXCL1, and IL-6.

26. The process of any of claims 19-25, wherein the polymer comprises at least one of poly(lactide-co-glycolide) (PLGA), CG910, CG950 or polytrimethylene carbonate (pTMC).

27. The process of any of claims 19-26, wherein the polymer comprises at least one of the first therapeutic agent or the second therapeutic agent incorporated therein.

28. The process of any of claims 19-27, wherein the first therapeutic agent is administered to the subject prior to the second therapeutic agent, and wherein the second therapeutic agent is administered to the subject after a predetermined duration from a start of the administering of the first therapeutic agent.

29. The process of claim 28, wherein the first therapeutic agent and the second therapeutic agent are administered to the subject systemically.

30. The process of claim 28, wherein predetermined duration is at least one day.

31. A process for preventing formation of, enlargement of, and/or rupture of an aneurysm in a subject comprising:
locating a device comprising a polymer at a site within a vasculature of the subject, wherein the polymer comprises a first therapeutic agent and a second therapeutic agent incorporated therein, and wherein the polymer is capable of controlled release of the first therapeutic agent and the second therapeutic agent,
wherein the first therapeutic agent comprises MCP-1 and/or osteopontin in an amount effective to promote recruitment of macrophages to the lesion site; and
wherein the second therapeutic agent comprises an inhibitor of at least one of IL-17A, CXCL1, or IL-6 effective to promote conversion of an amount of the macrophages to M2 phenotype macrophages at the site.

32. The process of claim 31, wherein the device is at least partially coated with a coating comprising the polymer, wherein the coating comprises an outermost layer comprising the first therapeutic agent and an inner layer comprising the second therapeutic agent.

33. The process of either claim 31 or 32, wherein the polymer wherein the polymer comprises at least one of poly(lactide-co-glycolide) (PLGA), CG910, CG950 or polytrimethylene carbonate (pTMC).

34. The process of any of claims 31-33, wherein the device is selected from the group consisting of a stent and an aneurysm coil.

35. The process of claim 31, wherein the device comprises an aneurysm coil.

36. The process of any of claims 31-35, wherein the first therapeutic agent comprises both MCP-1 and osteopontin.

37. The process of any of claims 31-35, wherein the second therapeutic agent comprises an inhibitor of IL-17A and an inhibitor of CXCL1.

38. The process of any of claims 31-35, wherein the second therapeutic agent comprises an inhibitor of each of IL-17A, CXCL1, and IL-6.

39. The process of any of claims 31-38, wherein the device is at least partially coated with the polymer.

40. The process of any of claims 31-38, wherein the device is at least partially formed from the polymer.

41. A device effective in treating a lesion at a lesion site in a subject comprising:
a polymer incorporating a first therapeutic agent and a second therapeutic agent therein, wherein the polymer is capable of controlled release of the first therapeutic agent and/or the second therapeutic agent;
wherein the first therapeutic agent comprises MCP-1 and/or osteopontin in an amount effective to promote recruitment of macrophages to the lesion site upon positioning of the device at the lesion site; and
wherein the second therapeutic agent comprises an inhibitor of at least one of IL-17A, CXCL1, or IL-6 effective to promote polarization of an amount of the macrophages to M2 phenotype macrophages upon positioning of the device at the lesion site.

42. The device of claim 41, wherein the device is at least partially coated with a coating comprising the polymer, wherein the coating comprises an outermost layer comprising the first therapeutic agent and an inner layer comprising the second therapeutic agent.

43. The device of either claim 41 or 42, wherein the polymer comprises at least one of poly(lactide-co-glycolide) (PLGA), CG910, CG950 or polytrimethylene carbonate (pTMC).

44. The device of any of claims 41-43, wherein the device is selected from the group consisting of a stent and an aneurysm coil.

45. The device of claim 44, wherein the device comprises an aneurysm coil.

46. The device of any of claims 41-45, wherein the first therapeutic agent comprises both MCP-1 and osteopontin.

47. The device of any of claims 41-45, wherein the second therapeutic agent comprises an inhibitor of each of IL-17A, CXCL1, and IL-6.

48. The device of any of claims 41-47 wherein the device is at least partially coated with the polymer.

49. The device of any of claims 41-47, wherein the device is at least partially formed from the polymer.

50. A composition effective in treating a lesion at a lesion site in a subject comprising:
a first therapeutic agent, a second therapeutic agent therein, and a pharmaceutically effective carrier,
wherein the first therapeutic agent comprises MCP-1 and/or osteopontin in an amount effective to promote recruitment of macrophages to the lesion site; and
wherein the second therapeutic agent comprises an inhibitor of at least one of IL-17A, CXCL1, or IL-6 effective to promote polarization of an amount of the macrophages to M2 phenotype macrophages at the lesion site.

51. The composition of claim 50, wherein the pharmaceutically effective carrier comprises a polymer capable of controlled release of the first therapeutic agent and/or the second therapeutic agent.

52. The composition of either claim 50 or 51, wherein the polymer comprises at least one of poly(lactide-co-glycolide) (PLGA), CG910, CG950 or polytrimethylene carbonate (pTMC).

53. The composition of any of claims 50-52, wherein the first therapeutic agent comprises both MCP-1 and osteopontin.

54. The composition of any of claims 50-52, wherein the second therapeutic agent comprises an inhibitor of IL-17A and an inhibitor of CXCL1.

55. The composition of any of claims 50-52, wherein the second therapeutic agent comprises an inhibitor of each of IL-17A, CXCL1, and IL-6.

56. A process of treating a lesion at a lesion site in a subject comprising:
administering an amount of MCP-1 and osteopontin to the subject effective to promote recruitment of macrophages to the lesion site; and
wherein the administering comprises releasing MCP-1 for a first predetermined duration followed by releasing osteopontin for a second predetermined duration.

57. The process of claim 56, wherein the first predetermined duration is 1-14 days and the second predetermined duration is 1-14 days.

58. The process of claim 56 or 57, further comprising administering an amount of a second therapeutic agent comprising an inhibitor of at least one of IL-17A, CXCL1, or IL-6 effective to promote polarization of an amount of the macrophages to M2 phenotype macrophages at the lesion site.

59. A composition effective in treating a lesion at a lesion site in a subject comprising:
MCP-1, osteopontin, and a pharmaceutically effective carrier, wherein the MCP-1 and osteopontin are combined with the pharmaceutically effective carrier such that MCP-1 is released for a first predetermined duration of time and osteopontin is released for a second predetermined duration of time.

60. The composition of claim 59, wherein the first predetermined duration is 1-14 days and the second predetermined duration is 1-14 days.

61. The composition of claim 59 or 60, further comprising an amount of a second therapeutic agent comprising an inhibitor of at least one of IL-17A, CXCL1, or IL-6.

62. A device comprising at least one aneurysm coil, wherein the aneurysm coil comprises at least one coating comprised of either a first composition comprising a polymer and a protein for recruiting macrophages to a lesion site of a vessel or a second composition comprising a polymer and a protein to promote polarization of an amount of the macrophages to M2 phenotype macrophages at the lesion site, or both.

63. The device of claim 62, wherein the first composition comprises a composition comprising a polymer and MCP-1 or a composition comprising a polymer and osteopontin, or composition comprising a polymer with both MCP-1 and osteopontin.

64. The device of claim 62 or 63, wherein the second composition comprises a composition comprising a polymer and at least one inhibitor of IL-17A, CXCL1 or IL-6, or a combination thereof.

65. The device of any of claims 62-64, wherein the at least one coating comprises a first coating comprising MCP-1 and/or osteopontin and a second coating comprising at least one inhibitor of IL-17A, CXCL1 or IL-6.

66. The device of any of claims 62-65, wherein the at least one coating further comprises a third coating comprised of a third composition comprising a polymer lacking a protein for recruiting macrophages to a lesion site of a vessel or a protein to promote polarization of an amount of the macrophages to M2 phenotype macrophages; wherein the third coating is positioned between the first and second coatings.

67. The device of any of claims 62-66, wherein the first coating is external to the second and third coatings.

68. The device of any of claims 62-67, wherein the at least one coating is comprised of one or multiple layers of the respective composition formed by one or multiple applications of the respective composition.

69. The device of claim 68, wherein the one or more multiple applications comprise dipping, spraying, rolling or brushing the device with the respective composition.

70. The device of any of claims 66-69, wherein the device further comprises a fourth coating comprised of a composition comprising a polymer and MCP-1 when the first coating is comprised of a composition comprising a polymer and osteopontin, or a composition comprising a polymer and osteopontin when the first coating is comprised of a composition comprising a polymer and MCP-1, and wherein the fourth coating is positioned between the first and third coatings.

71. A method for treating an aneurysm coil, the method comprising
applying to a surface of the coil at least one coating comprised of a first composition comprising a polymer and a protein for recruiting macrophages to a lesion site of a vessel, or a second composition comprising a polymer and a protein to promote polarization of an amount of the macrophages to M2 phenotype macrophages at the lesion site, or both.

72. The method of claim 71, wherein the first composition comprises a composition comprising a polymer and MCP-1 or a composition comprising a polymer and osteopontin, or composition comprising a polymer with both MCP-1 and osteopontin.

73. The method of either claim 71 or 72, wherein the second composition comprises a composition comprising a polymer and at least one inhibitor of IL-17A, CXCL1 or IL-6, or a combination thereof.

74. The method of any of claims 71-73 wherein the at least one coating comprises a first coating comprising MCP-1 and/or osteopontin and a second coating comprising at least one inhibitor of IL-17A, CXCL1 or IL-6.

75. The method of any of claims 71-74, wherein the at least one coating further comprises a third coating comprised of a third composition comprising a polymer lacking a protein for recruiting macrophages to a lesion site of a vessel and/or a protein to promote polarization of an amount of the macrophages to M2 phenotype macrophages; wherein the third coating is positioned between the first and second coatings.

76. The method of any of claims 71-75, wherein the first coating is external to the second and third coatings.

77. The method of any of claims 71-76, wherein the at least one coating is comprised of one or multiple layers of the respective composition formed by one or multiple applications of the respective composition.

78. The method of claim 77, wherein the one or more multiple applications comprise dipping, spraying, rolling or brushing the device with the respective composition.

79. The method of any of claims 75-78, wherein the device further comprises a fourth coating comprised of a composition comprising a polymer and MCP-1 when the first coating is comprised of a composition comprising a polymer and osteopontin, or a composition comprising a polymer and osteopontin when the first coating is comprised of a composition comprising a polymer and MCP-1, and wherein the fourth coating is positioned between the first and third coatings.

80. The device of any of claims 62-70, wherein the polymer comprises CG910 or CG950.

81. The method of any of claims 71-79, wherein the polymer comprises CG910 or CG950.
